(19) United States
(12) Patent Application Publication
(10) Pub. No.: US 2025/0177175 A1
(43) Pub. Date: Jun. 5, 2025

(54) Stent Delivery System

(71) Applicant: Micro Medical Solutions, Inc., Woburn, MA (US)

(72) Inventor: Gregory Sullivan, Woburn, MA (US)

(21) Appl. No.: 18/970,095

(22) Filed: Dec. 5, 2024

Related U.S. Application Data
(60) Provisional application No. 63/606,400, filed on Dec. 5, 2023.
(60) Provisional application No. 63/710,168, filed on Oct. 22, 2024.

(51) Int. Cl. A61F 2/95

(52) U.S. Cl. CPC A61F 2/95; A61F 2250/0058; A61F 2310/00149

(57) ABSTRACT

The present disclosure relates to a stent delivery system that is specifically designed for the treatment of chronic limb-threatening ischemia (CLTI) below the knee (BTK), through ankle, and to be utilized as a primary treatment or as a bailout treatment.

### CROSS REFERENCE TO RELATED APPLICATION

This application claims priority to and takes the benefit of Provisional Patent Application Ser. No. 63/606,400 filed on Dec. 5, 2023, and Provisional Patent Application Ser. No. 63/710,168 filed on Oct. 22, 2024, the contents of which are herein incorporated by reference.

### FIELD OF THE SYSTEM

The Instant system relates generally to medical devices, such as stents. In particular, the invention relates to a novel stent delivery system and method of manufacture thereof.

### BACKGROUND OF THE INVENTION

The prevalence of peripheral arterial disease (PAD) has been increasing in the developing world and is generally known that possibly greater than 6% of the population, 65 years and older, experiences some form of symptomatic PAD. The symptoms of BAD progress in severity from mild, intermittent claudication to chronic, limb-threatening ischemia (CLTI); where CITI is characterized by chronic resting leg pain, impaired wound healing and, eventually, amputation. Studies estimate that CLTI has an incidence of approximately 50 to 100 per 100, 000 people per year and is associated with mortality rates as high as 20% within the first 12 months, after onset.

Additionally, current studies state that within 1 year of diagnosis of CLII, 25% of patients will require major amputation and most of the remaining patients will have non-healed wounds. CITI patients may have disease in multiple levels and vessels, which requires multiple procedures to improve blood flow to the lower extremities. It is not uncommon for a patient to have subsequent procedures over the course of several weeks and months.

Additionally, patients encountered in the everyday CLTI normally have significant comorbidities (including Coronary artery disease, diabetes, history of peripheral intervention, history of amputation, history of cigarette smoking, heavily calcified lesions, and a high rate of chronic total occlusions). The worst-off individuals of this patient population are Rutherford Category 25 at baseline, with nonhealing ulcers and rest pain patients and some even exhibit severe claudication.

Unlike other vascular stent procedures, CITI patients may have multilevel disease and vessels that require multiple procedures to improve blood flow to the lower extremities.¹³ Revascularization remains the cornerstone of therapy for CLTI and is recommended by the professional guidelines. To date, plain old balloon angioplasty (POBA) is still an important treatment method in CLTI. However, POBA is limited by the occurrence of recoil and severe flow-limiting dissections. Stenting-more specifically, the use of drug-eluting balloon-expandable stents-is feasible in short lesions, but is limited by the need to use multiple stents to achieve full lesion coverage and dissection repair. Thus, there is the need for other treatment options, such as longer devices that do not restrict vessel-wall compliance and flexibility, as much as the traditional balloon-expandable devices do.

Endovascular techniques to treat claudication include balloon dilation (percutaneous transluminal angioplasty [PTA]), stents, and atherectomy. These techniques continue to evolve and now include covered stents, drug-eluting stents, cutting balloons, and drug-coated balloons. The technique chosen for endovascular treatment is related to lesion characteristics (eg, anatomic location, lesion length, degree of calcification) and operator experience. PIA depends upon mechanical dilation of the artery and is associated with plaque fracture, intimal splitting, and localized medial dissection.

A stent is an elongated device used to support an intraluminal wall. Stenosis is an abnormal narrowing in a blood vessel or other tubular organ ox structure. The narrowing prevents the valve from opening fully, which obstructs blood flow from the heart and onward to the rest of the bod There exist a wide variety of stents used for different purposes depending on the type of narrowing vessel to the body. As used herein, the term “stent” is shorthand reference referring to the wide varieties of stents, both covered stents or uncovered stents.

Stents are typically implanted within the vascular system to reinforce collapsing, partially occluded, weakened or under dilated sections of vessel and valves. Stents have also been successfully implanted In urinary tracts and bile ducts to reinforce those body vessels. This invention is applicable in all of these situations,

In general, the typical procedure for implanting a self-expanding stent is to first open the region of the vessel with balloon catheter and then place the stent in a position bridging the weakened the portion of the vessel.

Localized post-PTA dissection is a common and expected adverse outcome associated with the angioplasty mechanism. Following PTA, many physicians place metal stents to maintain luminal patency, which improves blood flow, or to bail out failed PTA due to flow-limiting dissections, acute vessel recoil, and persistent residual stenosis. Stents used in the lower leg are typically either self-expanding nickel-titanium alloy (nitinol) or balloon-expandable systems. Positioning of the stent may be followed by the technique wherein a separate balloon catheter is positioned within the stent and expanded to radially expand the stent for implantation.

The safety and effectiveness of stents in the vasculature is well established in both coronary and peripheral vessels above the knee. Several studies have also evaluated the utility of stents for use in infrapopliteal lesions, but there is still a lack of data regarding what kind of stent(s) should be used and a treatment algorithm has not been established.

### SUMMARY OF THE INVENTION

The instant apparatus and system, as illustrated herein, is clearly not anticipated, tendered obvious, or even present in any of the prior art mechanisms, either alone or in any combination thereof. A versatile system, method and series of apparatuses for creating and utilizing a series of specialized micro mechanisms as part of a stent delivery device and other like systems disclosed,

In one aspect, the present apparatus introduces a manner in which to remove atherosclerosis from blood vessels and recanalize arteries, through intervention below the knee, within the ankle or below the ankle of the patient.

It is an object of the instant system to introduce radiopaque medical device, such stent, for use or implementation in a body lumen. In a preferred embodiment, the stent may be used in patients with critical limb ischemia (CLI), below-the-knee revascularization.

It is an additional object of the instant system to introduce an interruptive concept of stenting at the level of the ankle strap area, which includes an extension of a stent from above the ankle to below the ankle, including the pedal circulation.

It is an additional object of the instant system to introduce a radiopaque medical device which vitiates ankle strap crush effect and overcomes areas of high stress, torsion, extension, and body weight considerations, in below the ankle procedures.

It is a further object of the instant system to introduce a unique liner located internally within the stent delivery system which exhibits two (2) or three (3) times the tensile and radial strength as a hypotube liner and provides more lubricity using the same diameter.

It is an object of the instant system to introduce a unique liner located internally within the stent delivery system to convert finesse catheter (utilized only by highly skilled physician level operators) into robust mechanism which is 2-3 times stronger 30 by 8 catheter 2-6, 10 wire to get into the foot, use 150 2-3 times stronger so you don't have to have experts to preform procedures.

In recent years, for the treatment of critical limb ischemia, many medical professionals have investigated employment of a method know as endovascular intervention. Like any evolving treatment, utilization of endovascular intervention poses multiple challenges including issues with lesions formed from occlusions, or blockage in a canal, vessel, or passage of the body. Retrograde pedal/tibial artery access, in conjunction with antegrade access proves to recanalize impaired tibial vessels.

In an additional preferred embodiment, a radiopaque medical device is constructed from a tubular-shaped body having a thin wall defining a specific strut pattern. In an additional aspect, the present apparatus introduces a new radiopaque medical device, such as a stent, wherein the tubular body includes a super elastic, nickel-titanium (nitinol) alloy.

In one embodiment, the alloy may further include an additional element of platinum. In an additional embodiment, the alloy may further include an additional element selected from the group of chemical elements consisting of iridium, gold, tungsten, rhodium, tantalum, silver, etc.

In one embodiment; the system as disclosed herein is particularly suited for transpedal and tibiopedal retrograde revascularization in PAD patients. Transpedal is an interventional approach that can be used to treat patients with advanced peripheral artery disease (PAD) or critical limb ischemia (CLI). The instant method may involve making a cutdown and puncturing the arteries in the feet to create a blood flow channel through the blocked vessel. In the instant scenario, ultrasound guidance is used to access the tibiopedal vessels.

In one aspect the present apparatus introduces a novel medical device, such as a stent, wherein the stent is highly radiopaque. In a further aspect, the present apparatus introduces a new radiopaque medical device, such as a stent, wherein the stent does not require multiple layers or coatings of both nitinol and a radiopaque material. Rather, the radiopaque device, such as a stent, wherein the body of the stent comprises one alloy of combined nitinol and radiopaque material such as platinum.

Additionally, the present apparatus introduces a new radiopaque medical device, such as a stent, wherein the stent is self-expanding. The self-expanding stent may be loaded within a delivery System. When loaded, the stent will be collapsed and compressed and then released and automatically expanded at the point of delivery. Herein, the stent may be designed to perform various mechanical functions within a lumen.

In one embodiment, the radiopaque, self-expanding stent may be used in patients with critical limb ischemia (CLI), for below-the-knee revascularization. Because the preferred embodiment is to utilize the stent in patients with critical limb ischemia, it is an additional embodiment that the additional element alloyed with the nitinol does not diminish the super elastic characteristics of a nitinol stent.

To achieve sufficient degree of radiopacity and still ensure that the super elastic properties of the stent stay true, in one aspect, the present apparatus, includes stent wherein the platinum additional element's atomic percent is greater than or equal to 2.5 and less than or equal to 15. With these characteristics, the platinum still comprises high radiopacity but does not inhibit the flexibility and elastic qualities of the nitinol.

In yet another aspect, the present invention introduces a method for providing a radiopaque nitinol-platinum stent. In preferred embodiment, the method entails providing a tubular-shaped body having a thin wall, wherein the body includes a super elastic nitinol alloy and the alloy further includes an additional element such as platinum. In further embodiments, the additional element may also be selected from the group of chemical elements consisting of iridium, gold, rhenium, tungsten, palladium, rhodium, tantalum, silver, ruthenium, or hafnium.

The method further includes forming a strut pattern; wherein the stent is highly radiopaque. The step of providing a tubular-shaped body includes melting nickel, titanium, and the ternary element and cooling the mixture to form an alloy ingot, hot forming the alloy ingot, hot or cold forming the alloy ingot into a cylinder, drilling the cylinder to form tubing, cold drawing the tubing, and annealing the tubing.

Realizing one aspect of the present invention relates to a new medical device, such as a stent, that is biocompatible, radiopaque and MRI compatible. In another embodiment, a radiopaque and MRI compatible medical device, such as a stent, is constructed from a tubular-shaped body having a thin wall defining a strut pattern; wherein the tubular body includes a nitinol alloy, and the alloy further includes the additional a quantity of the element platinum. One embodiment may include nickel and titanium plus either platinum.

The alloy having a composition of the present invention can be used with any medical device, especially medical devices that require either radiopacity or MRI compatibility. In some embodiments, these ternary elements depress the transformation temperature such that no stress induced martensite is formed at body temperature.

In one embodiment, a braided woven stent in the body utilizing a combination of nitinol tube w/ a small core of platinum for both for deployment/delivery purposes in the body and diagnostic applications. Stent has been designed specifically for usage in procedures below the knee, i.e. for the anterior tibial, posterior tibial, and peroneal arteries of the lower leg. We are also targeting the pedal artery that runs in the foot but we have not conducted the testing as of yet.

In one embodiment, the stent will be used in conjunction with balloon angioplasty and/or other interventional procedures such as atherectomy, Atherectomy is a minimally invasive endovascular surgery technique for removing atherosclerosis from blood vessels within the body. It is an alternative to angioplasty for the treatment of peripheral artery disease, with no evidence of superiority to angioplasty. The stent will be deployed after these procedures to provide a scaffold to maintain stent patency. With the scaffold in place, restoration of blood flood could avoid the need for amputation.

The utilization of platinum increases visualization of the stent both during deployment and post procedural. Thus, platinum primarily is used for enhancement under fluoroscopy but is also highly visible by way of ultrasound. The use of ultrasound could ultimately move the procedure from the catheterization laboratory, (“cath lab”) to a physician's office.

In terms of deployment and use in the body, in one embodiment; the stent is utilized to enter the body below the knee and then wrap around the ankle while using ultrasound for detection and positioning, a pedal/tibial access (at the ankle) and proceeding retrograde to deploy the stent in the lower leg. Regarding deployment of the stent in the foot, the same access point may be utilized, however, the physician would proceed antegrade.

In many scenarios, the retrograde approach, especially when entering sufficiently close to the occlusion, allows for successful crossing of the occlusion, with a very low rate of occlusion at the access point of the pedal/tibial vessel. Accessing the pedal/tibial vessels in retrograde fashion is normally more successful than the antegrade approach, however, with the additional usage of an ultrasound assist with the instant radiopaque system, much greater success rate in getting the apparatus through the occlusion may be exhibited within antegrade scenarios,

In one embodiment, fluoroscopy may be utilized in order to assist the administering physician. Fluoroscopy is a type of medical imaging that shows a continuous medical X-ray image on a monitor, much like an X-ray movie, to obtain real-time moving images of the interior of an object. During fluoroscopy procedure, an X-ray beam is passed through the body allowing physician to observe the internal structure and function of a patient, in other words both the anatomy and physiology of a patient.

In another embodiment, ultrasound may be utilized in order to assist the administering physician. Ultrasound may be defined as frequencies higher than the upper audible limit of humans hearing range. Ultrasound is no different from “normal” (audible) sound in its physical properties, except in that humans cannot hear it. This limit varies from person to person and is approximately 20 kHz (20,000 hertz) in healthy, young adults.

Ultrasound devices operate with frequencies from 20 kHz up to Several gigahertz. Ultrasound is used in many different fields. Ultrasonic devices are used to detect objects and measure distances, Ultrasound imagining or sonography is often used in medicine. Medical sonography (ultrasonography) is an ultrasound-based diagnostic technique used to visualize muscles, tendons, and many internal organs, to capture their size, structure and any pathological lesions with real time tomographic images.

There has thus been outlined, rather broadly, the more important features of a medical device delivery system for protection during surgery embodiments in order that the detailed description thereof that follows may be better understood, and in order that the present contribution to the art may be better appreciated. There ate additional features of the invention that will be described hereinafter and which will form the subject matter of the claims appended hereto.

In this respect, before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

These together with other objects of the invention, along with the various features of novelty, which characterize the invention, are pointed out with particularity in the claims annexed to and forming a part of this disclosure. For a better understanding of the invention, its operating advantages and the specific objects attained by its uses, reference should be made to the accompanying drawings and descriptive matter in which there are illustrated preferred embodiments of the invention.

### BRIEF DESCRIPTION OF DRAWINGS

Advantages of the present system, apparatuses and methods will be shown below and a better understanding will be apparent from the following detailed description of exemplary embodiments thereof, which description should be considered in conjunction with the accompanying drawings, in which:

FIG. 1 is a longitudinal view of the novel Microstent® peripheral delivery system and further illustrating an exploded view longitudinal cross-sectional view of the stent;

FIG. 2 is a longitudinal view of the novel Microstent® peripheral delivery shaft and hub assembly;

FIG. 3 is an exploded longitudinal view of the stent loaded within the peripheral delivery system;

FIG. 4 is a longitudinal side view of the novel braided 3.2f Microstent® delivery shaft of the Microstent® peripheral delivery system illustrating one or one marker bands 60, the marker band adhesive layer 70, the shaft Liner 80, the braided skeleton tube 90 and an extrusion delivery sheath; and a stent.

FIG. 5 is front view of the view of the novel braided 3.2f microstent delivery shaft illustrating one or one marker bands, the marker band adhesive layer, the shaft PTFE etched liner, the braided skeleton tube, an extrusion delivery sheath and a stent the delivery shaft.

FIG. 6 is perspective view of the braided skeleton tube Construction of the shaft.

FIG. 7 illustrates the processes of balloon placement and balloon dilation.

FIG. 8A illustrates the novel generation 2, or GEN 2 stent delivery system,

FIG. 8B illustrates the generation 1, or GEN 1 stent delivery system.

FIG. 9 illustrates a comparison of inner lumen of a state of the art delivery system and toner lumen of an instant novel delivery system.

FIG. 10 illustrates durability of an instant GEN 2 stabilizer Inner lumen, compared to that of a GEN 1 lumen.

FIG. 11 illustrates abrasion results of scratched polyimide.

FIG. 12 illustrates Stabilizer Tracking Forces generated by GEN 1 stent delivery system and GEN 2 on Guidewire.

FIG. 13 illustrates a set of comparative inner lumen strength graphs.

FIG. 14 illustrates braided delivery sheath internal reinforcement from Gen 1 to Gen 2.

FIG. 15 illustrates a graphical representation of a delivery sheath shaft tensile.

FIG. 16 illustrates a delivery sheath robustness.

FIG. 17 illustrates Hub Sub Tensile strength for Gen 2 system versus the GEN 1 system.

FIG. 18 illustrates a GEN 2 delivery system.

### DETAILED DESCRIPTION OF DRAWINGS

The present system pertains to improved medical devices providing advantages in precision, strength and other desired properties. Accordingly, an illustrative but non-limiting example of the present system may be found in a medical device such as a stent delivery system that is design to work in conjunction with the MicroMedical BF stent and delivery system.

In one embodiment, the stent, which herein illustrated for optimal use is a MicroStent® intended for permanent implantation and may be comprised of a self-expanding nitinol stent, designed to be preloaded into a 3.2-Fr, 0.014″, over-the-wire delivery system. The device may be intended to improve luminal diameter in the treatment of ischemia in the lower leg with reference vessel diameters (RVDs) from 2.0 mm to 4.5 m. The MicroStent® (40-cm delivery system) and the MicroStent XL® (120-cm delivery system) may be formed from nitinol wires woven in a braided configuration.

Upon deployment, constant, gentle outward force to establish and maintain the luminal diameter. The stent wires have radiopaque platinum core that provide improved visibility for the braided stent during deployment and Subsequent follow-up. The delivery system includes a 3.2-Fr sheath catheter with a coaxial inner assembly (stent stabilizer)

The system may also include a proximally located, rotational hemostasis valve on the sheath catheter may provide hemostasis and a safety lock may be deployed in order to prevent premature deployment of the stent as well as a system to irrigate the catheter.

Next, the stent stabilizer may terminate distally through the preloaded stent and out the distal end of the sheath catheter. The distal portion of the sheath catheter may contain a radiopaque marker band.

A second radiopaque marker band may be located on the stabilizer to mark the proximal portion of the self-expanding stent when it is positioned within the space between the stent stabilizer and the sheath catheter. The stent is positioned at the target site using at least one radiopaque marker bands. To one embodiment, two radiopaque marker bands may be utilized. In this embodiment, one marker band may be located distal to the stent and the additional marker band may be located proximal to the stent.

In an additional embodiment, the stent's braided structure may also be composed of a radiopaque material. And such an embodiment, for proper stent implantation, proper lesion characterization using standard techniques such as angiography of intravascular ultrasound may be utilized. Along these lines, the target lesion should be predilated with one (1) or more balloons (with increasing outer diameter inflation) to achieve vessel diameter equal to the diameter of the herein described stent mechanism, and with longer inflation times recommended (˜1-2 minutes).

When selecting the stent size, preferably the Stent:RVD ratio should match 1:1. For example, if the proximal RVD is 3.5 mm, the distal RVD is 3.0 mm and the average RVD is 3.25 m, the 3.5-mm diameter stent should be selected. For example, if treating an 80-mm lesion, the stent size selected should fully cover 80 mm plus approximately 5-10 mm of healthy intima proximal and distal to the lesion per the stent dimensions table.

In order to achieve successful deployment of the MicroStent, device deployment should be slow and steady to avoid elongation and stacking, which can reduce the designed engineering properties of the MicroStent. For lesions requiring multiple MicroStents, a ˜1 cm overlap is recommended. Deployment should always be distal to proximal (anatomically) such that the proximal stent (upstream) lays within the distal stent (downstream).

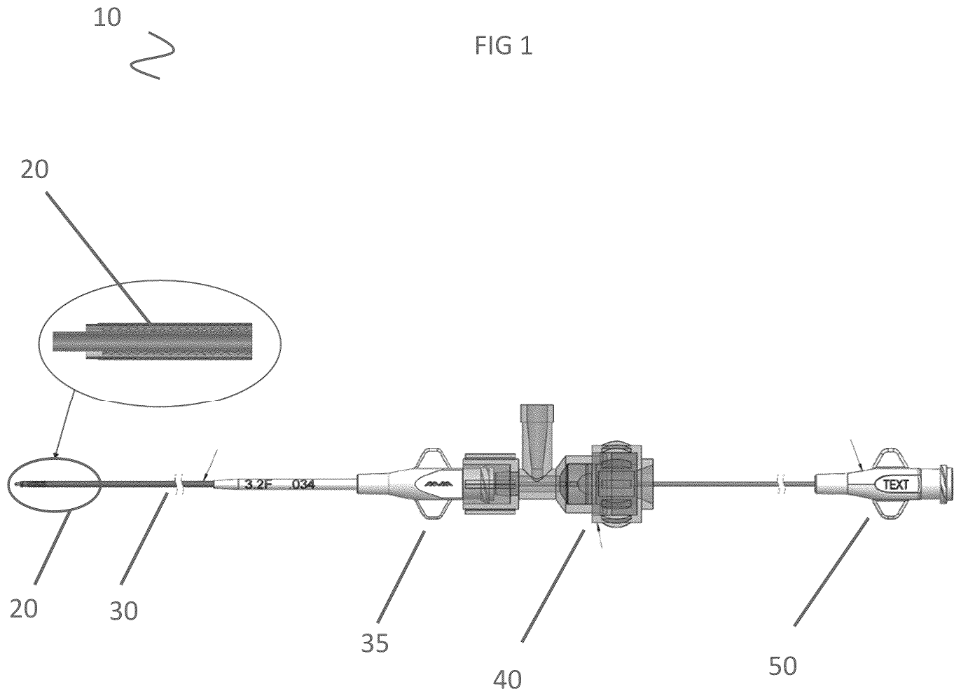

FIG. 1 is a longitudinal view of the novel microstent peripheral stent system 10 including the delivery sheath 30 and hub assembly 35 a stent stabilizer 50 and Tuchy Borst device 40, a stent device 20 and further illustrating an exploded view longitudinal cross-sectional view of the stent 20.

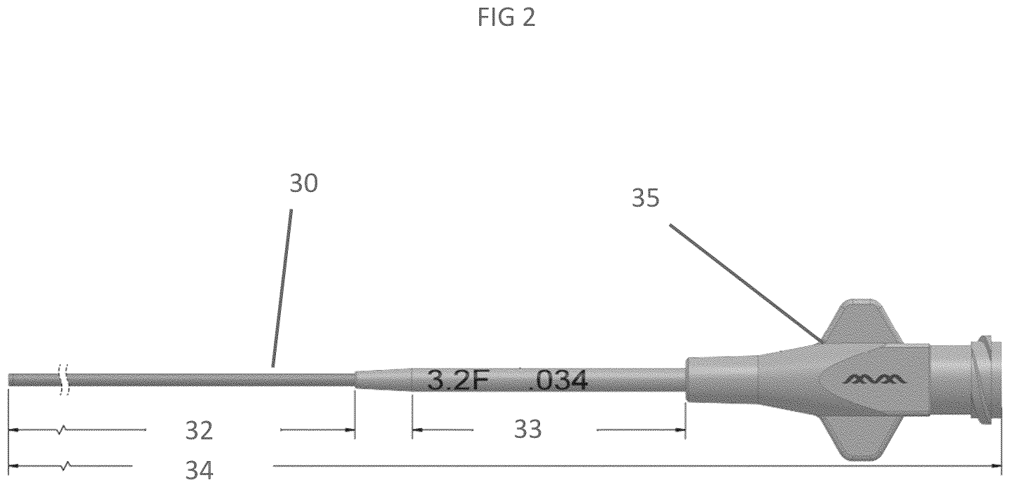

FIG. 2 is a longitudinal view of the novel microstent peripheral delivery system including the delivery sheath 30 and hub assembly 35. The Working Length 32, Reflow Length 33 and Overall length 34 are additionally illustrate therein. START HERE

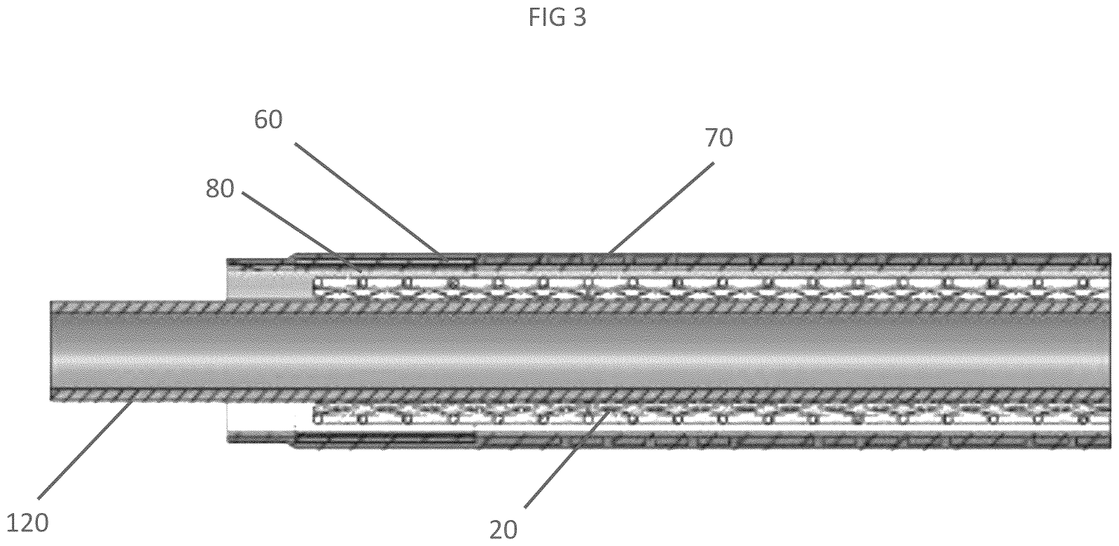

FIG. 3 is an exploded cutaway longitudinal view of the delivery shaft 30 of the peripheral delivery system with the stent loaded within the peripheral delivery system, further illustrating one or one marker bands 60, the marker band adhesive layer 70, the shaft Liner 80, the braided skeleton tube 90 and an extrusion delivery sheath 30, the stent 20 and the guide wire 120.

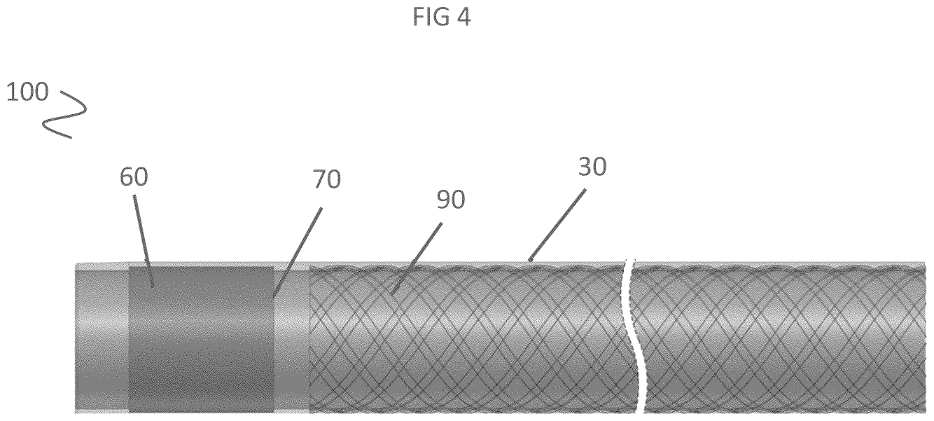

FIG. 4 is a longitudinal side view of the novel braided 3.2f microstent delivery microstent shaft 100 of the peripheral delivery system 10 illustrating one or one marker bands 60, the marker band adhesive layer 70, the shaft Liner 80, the braided skeleton tube 90, an extrusion delivery sheath and a stent 20.

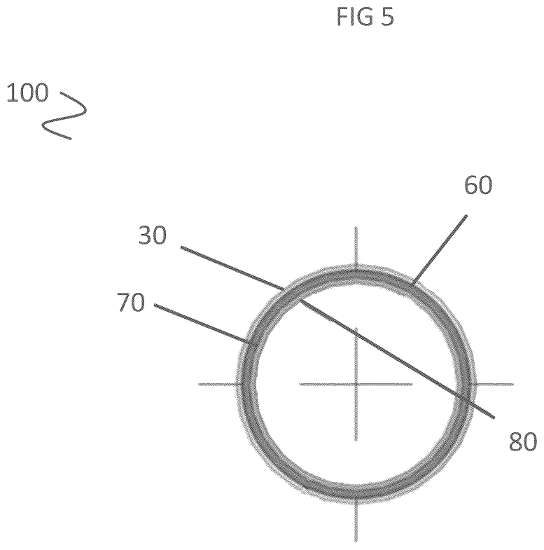

FIG. 5 is front view of the view of the novel braided 3.2f microstent delivery shaft 100 illustrating one or one marker bands 60, the marker band adhesive layer 70, the shaft Liner 80, the braided skeleton tube 90, an extrusion delivery sheath 30 and a stent 20. the delivery shaft 30.

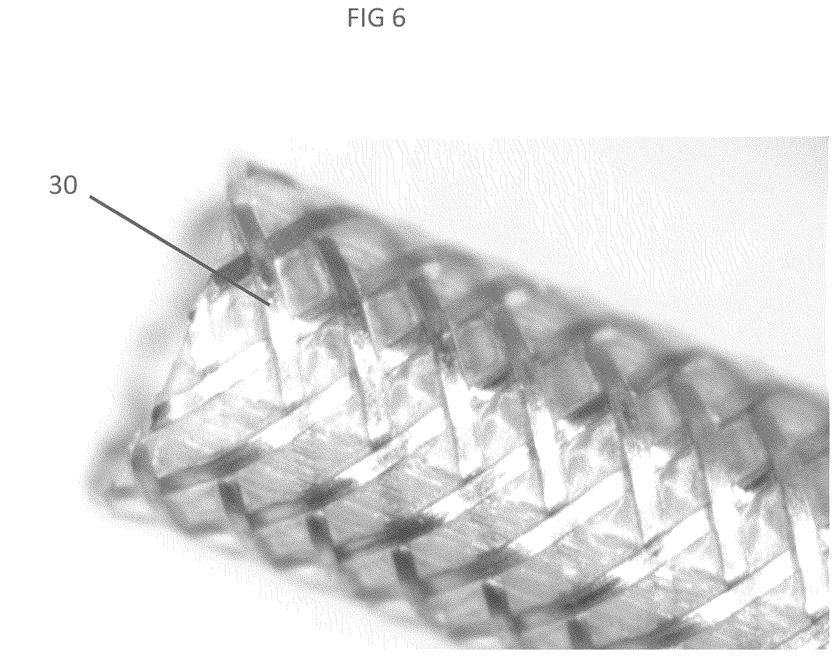

FIG. 6 is perspective view of the braided skeleton tube adhesive layer 70, the shaft Liner 80, the braided skeleton tube 90 construction of the shaft.

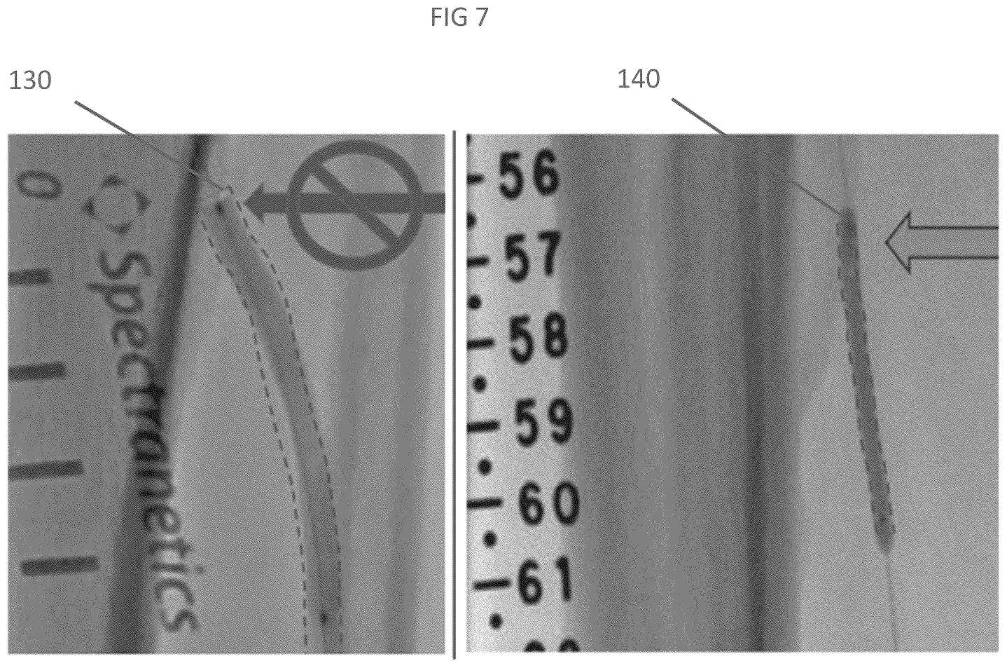

After completion of stent deployment, post dilation strongly recommended. The labeled diameter of the balloon used should not exceed the diameter of the stent. The operator should ensure that both the distal and proximal ends of the stent are adequately dilated. The balloon should extend just outside of the stent end to ensure full dilation as illustrated in FIG. 7.

FIG. 7 illustrates balloon dilation and placement. In one embodiment, the balloon marker band may be placed just outside of the stent ends to fully dilate. The red arrow (left panel) demonstrates incorrect placement and dilation of the balloon 140 and the green arrow (right panel) demonstrates correct placement and dilation of the balloon 150.

In some instances, postprocedural dual-antiplatelet therapy (also called DAPT) should be given per industry best practices after endovascular revascularization for lower-extremity PAD. In summary: appropriate lesion characterization, vessel preparation, stent size selection, and DAPT are critical for optimal outcomes. Dual antiplatelet therapy is a treatment to help stop harmful blood clots from forming. This involves taking 2 types of antiplatelet medicines. One of these medicines is usually ASA (aspirin) and the other is a special type of medicine called a P2Y12 Inhibitor.

Patient characteristics and lesion characteristics detailed in include a total of 77 patients were enrolled across 9 sites in 5 European countries representing 78 lesions and a total of 91 MicroStent devices. Addressing the capabilities of the instant system, in patients treated with a median body mass index was 25.5 kg/m (range, 2.2-38.3) and Rutherford category ranging from 3 to 6. The majority of patients presented with Rutherford category 5 (68.8%) followed by Rutherford 4 (19, 5%), Rutherford 3 (9.1%), and Rutherford 6 (2.6%). The majority of patients suffered from diabetes (75,3%), history of coronary artery disease (29.9%) history of peripheral intervention (45.9%); and history of amputation (21.6%). Slightly more than 39.0% were former smokers, 13.0% were current smokers, and 48.1% were nonsmokers.

The median target-lesion length was 45 mm (range, 10-400 mm) and 51.9% were chronic total occlusions. The majority of lesions were located in the anterior tibial artery (38.5%), followed by tibial-peroneal trunk (28,2%), posterior tibial artery (17.9%), peroneal artery (9,0%), popliteal artery (2.6%)/superficial femoral artery (1.3%), common plantar artery (1.3%), and distal popliteal artery/proximal anterior tibial artery (1.3%).

Calcified lesions were found in 40.3% of patients; 19.5% of them were severely calcified. In 85.9%, de novo lesions were treated (restenotic lesions, 14.1%). The rate of adjunctive therapies used during target-lesion treatment was 14.1%, with drug-coated balloons used in 45.5% of cases, followed by specialty balloon (e.g., scoring, cryotherapy) used in 45.5% of cases and drug-eluting stents used in 9.1% of cases. Out of 77 patients treated with the MicroStent®, 84.4% were implanted with 1 stent, followed by 13.0% implanted with 2 stents, and 2.6% implanted with 3 stents. A total of 52.2% of MicroStents were implanted for primary treatment of the target lesion and 47.8% were implanted as bailout options after failed treatment.

The majority of the reasons for bailout were flow-limiting dissection (grade C or higher) ox vessel perforation (59.5%); followed by persistent residual stenosis ≥30% (21.4%), and acute vessel recoil or other negative occlusive complications (19,0%); the reason was not indicated in 0.2%. Pre-dilation was performed in all patients prior to deployment of the MicroStent®, while post dilation was performed in 88.9% of implanted MicroStent®.

In accordance with this system, method and set of accompanying apparatuses, there is provided a stent delivery system comprising a stent stabilizer and Tuohy Borst device. Tuohy Borst adapters are medical devices used for the advancement of catheters or optical fibers from 0 to 6 FR while preventing the backflow of body fluids. The Tuohy Borst apparatus is used to prevent the backflow of fluid around an instrument inserted through the working channel of flexible and rigid ureteroscopes. Tuohy Borst adapters form a leak-proof seal around instruments like catheters and optical fibers, making them suitable for a wide variety of interventional and diagnostic procedures.

The Tuohy Borst adapter enables access and placement of devices while helping to reduce backflow of gas or fluids. These attributes render the Tuohy Borst adapter particularly well suited for a wide range of interventional applications as the Tuohy Borst adapter assists in preventing blood loss from OEM catheters.

The employment of a Tuohy-Borst adapter for minimally invasive surgical procedures may provide numerous advantages over open surgery, including the need for smaller incisions, shorter recovery times and reduced pain and discomfort. Such minimally invasive procedures often involve endoscopy and the introduction of catheters through small incisions. for example, such minimally invasive procedures are used to address a wide variety of vascular problems, including atherosclerosis, aneurysms, varicose veins, vascular malformations, blood vessel blockage due to stroke, etc. many of these treatment procedures involve the use of catheters, including, but not limited to, angioplasty with balloon-tipped catheters, vascular stenting, and embolization.

The introduction of catheters and endoscopic devices can lead to the issues regarding the backflow of body fluids. Additionally, some of these procedures require the simultaneous infusion saline and the insertion of the catheter. These concerns are addressed using a backflow adapter such as a Tuohy Borst adapter that allows for the introduction of various devices of different girths and prevents backflow by forming a hermetic seal. In one embodiment, a Tuohy-Borst y-connector subcutaneous valve manufactured from artificial materials for implantation may be utilized. Furthermore, in the creation of the system, no laser cutting ox welding of any metal pieces need be utilized. The only laser process is laser ablating some holes in the stabilizer polymer shaft for adhesive application.

In one embodiment, the stent delivery system may comprise at least one marker band 60, at least one marker band adhesive layer 70, a shaft liner 80, braided skeleton tube 90, an extrusion delivery sheath; and a stent. In one embodiment, the stent delivery system may include a delivery sheath formed on a mandrel and the delivery sheath comprises 3.2F construction.

Additionally, the stent delivery system may include an: Platinum/Iridium embodiment wherein the marker band comprises alloy and in one embodiment the Marker band nay comprise Platinum/Iridium alloy in a ration of Pt 90/Ir 10 alloy.

Also, the marker band adhesive of the stent delivery system may contain Cyanoacrylate Loctite 4014 or other such materials. Additionally, the shaft liner 80 of the stent delivery system may be manufactured from Polytetrafluoroethylene (PTFE). PTFE is a synthetic fluoropolymer of tetrafluoroethylene. Being hydrophobic, non-wetting, high density and resistant to high temperatures, PTFE is an incredibly versatile material with a wide variety of applications. Moreover, PTFE exhibits excellent non-stick properties.

Additionally, the braided skeleton tube 90 may be constructed with comprises 304 SS flatwire and an aramid fiber which for this particular application may be selected from Technora, Vestamid and others as known to the art. Technora is an aramid that is useful for variety of applications that require high strength ox chemical resistance.

Aramid fibers, short for aromatic polyamide, are a class of heat-resistant and strong synthetic fibers. They are used in aerospace and military applications, for ballistic-rated body armor fabric and ballistic composites, in marine Cordage, marine hull reinforcement, as an asbestos substitute,¹¹¹ and in various lightweight consumer items ranging from phone cases to tennis rackets. The chain molecules in the fibers are highly oriented along the fiber axis. As a result, higher proportion of the chemical bond contributes more to fiber strength than in many other synthetic fibers, Aramids have a very high melting point (>500° C.).

Additionally, VESTAMID® Stands for an entire range polyamide with custom tailored properties. Evonik obtains the desired characteristics in the materials by chemical modification of the base polymer, or physical modification—by incorporation of glass fibers, Teflon, or graphite, for example—or a combination of both. In this way Evonik is able to meet virtually any customer requirement with an extremely wide range of VESTAMID® grades

In one embodiment, the stent delivery system may contain a delivery sheath 30 constructed of extruded nylon Vestamid® care ML21, natural. The stent delivery system may also feature a marker band 60 which may be fabricated from a Platinum/Iridium alloy, and in one embodiment, the Platinum/Iridium alloy may be apportioned in a ration of Pt 90/Ir 10 alloy.

In line with the use of the marker band 60, The stent delivery system 10 may utilize an accompanying adhesive, which may be Cyanoacrylate, Loctite 4014. In one embodiment, the stent delivery the marker band comprises a 3.2F construction. The stent delivery system's delivery shaft includes a braided construction. The stent delivery system's marker band includes a 3.2F construction. The stent delivery system also has a Hub assembly. The stent delivery system's hub assembly includes a 3.2F construction.

Herein the Generation 2, or GEN 2 delivery system provides a top-level system by affording the advantages of the enhanced stabilizer with the novel inner lumen, working in conjunction with the advantages of afforded by the braided delivery sheath, shown above. Overall delivery system improvements and goals include improved pushability and durability thru manufacturing improvements alone. In addition, the instant enhanced system retains all of the system requirements/parameters and product specifications, while achieving increased operational and technical capabilities. Moreover, within the instant iteration, the system introduced is markedly more manufacturable, less vulnerable to supply chain risk and less expensive than concurrent systems.

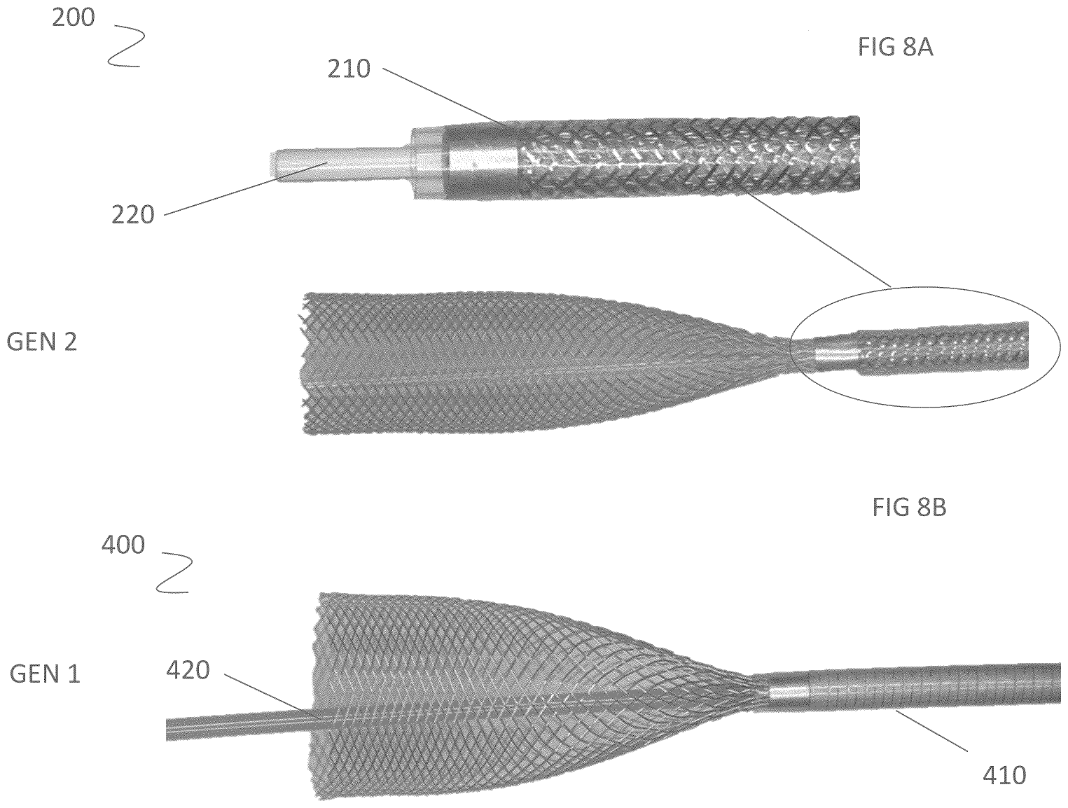

FIG. 8A illustrates the novel generation 2, or GEN 2 stent delivery system 200, further illustrating the inner lumen 210 working in conjunction with the braided delivery sheath 220. FIG. 88 also illustrates the generation 1, or GEN 1 stent delivery system 400, and further illustrates the GEN 1 inner lumen 410 working in conjunction with the GEN 1 delivery sheath 420.

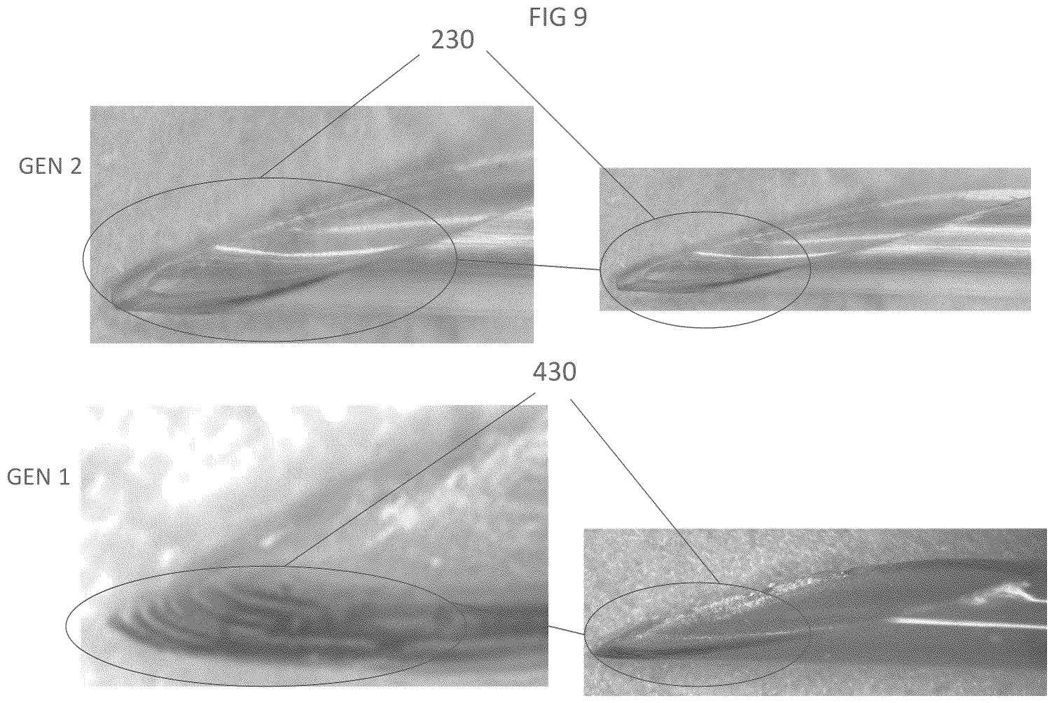

FIG. 9 illustrates a comparison of the inner lumen 420 of the state of the art delivery system 400 and the inner lumen 220 of the instant novel delivery system 200 to illustrate structural differences. To FIG. 9, both the inner lumen 420 of the state-of-the-art delivery system 400 and the inner lumen 220 of the instant novel delivery system 200 are shown as cross sections out at a sixty degree (60°) angle, in order to illustrate the structural differences between single solid layer construction 230 of the GEN 2 system, which is extruded then drawn to final shape, of the new lumen construction versus the multilayer dispensed construction 430 of the GEN 1 instant system.

Addressing the improved lumen attributes, in many cases, the state-of-the-art stabilizer inner lumen construction, under the industry standard, comprises high strength precision tube constructed with multiple layers in a dispensed environment. The instant system may comprise a single solid layer, which in one embodiment may be extruded, then drawn to the final shape of the lumen. In one embodiment, a newly developed process to create a high strength precision tube is described. In one embodiment, nylon may be the material of choice which may be drawn to meet the shape and requirements of the lumen,

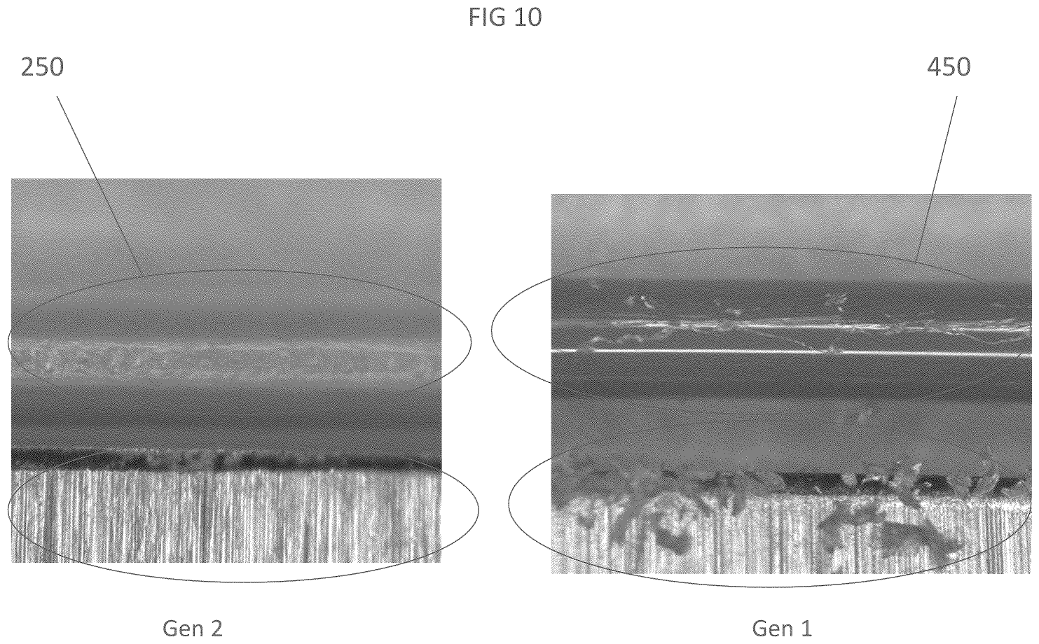

FIG. 10 illustrates the durability of the instant GEN 2 stabilizer inner lumen, versus that of the GEN 1 lumen in state of the art to date. The results of an abrasion test including five times (5×) passes w/single edge razor at forty-five degree (45°) angle illustrate that the instant stabilizer inner lumen 200 exhibited no layer delamination 250, whereas the state-of-the-art stabilizer inner lumen 400 exhibited substantial layer delamination 450.

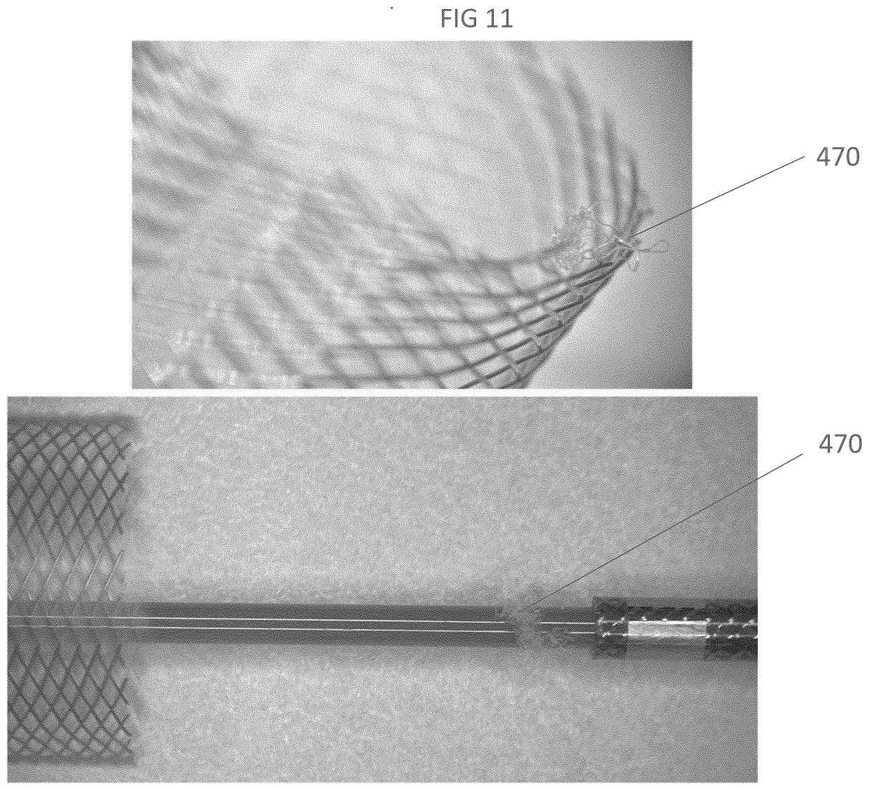

FIG. 11 further illustrates similar results as the GEN 1 lumen illustrates abrasion concerns which yielded scratched polyimide 470 lodged to the stent end, as well as scratched polyimide connected to device.

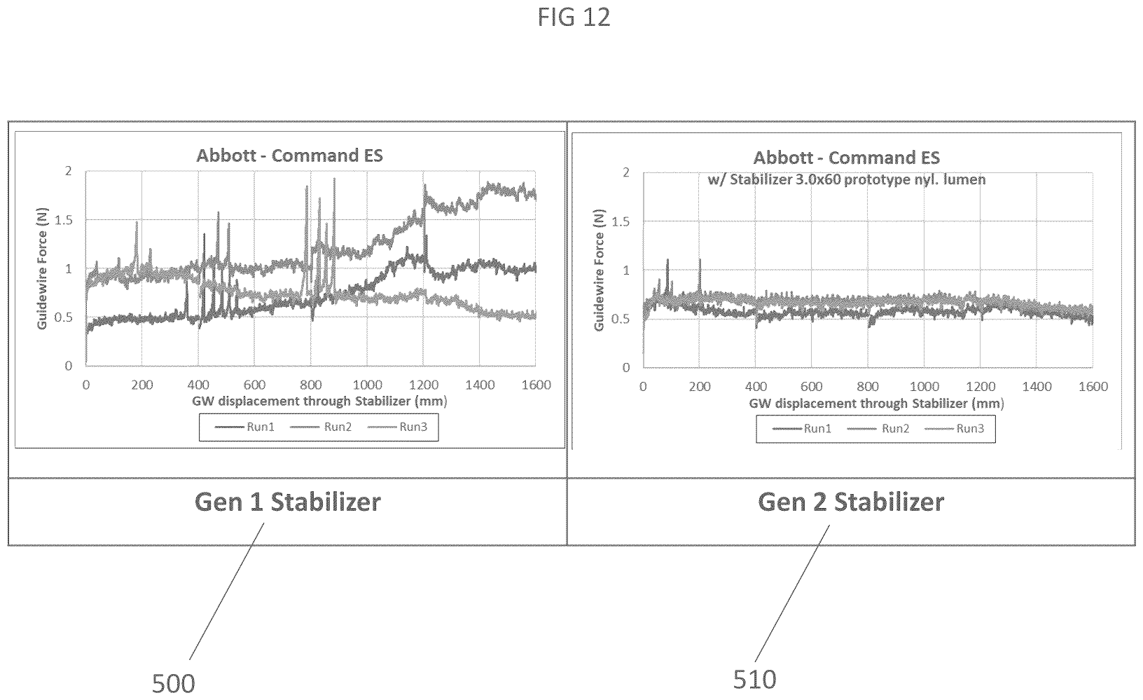

FIG. 12 illustrates the Stabilizer Tracking Forces generated by the GEN 1 stent delivery system 500 and the GEN 2 510 set on a guidewire utilizing an Abbott Command ES involved w/ CDE 21-000, Constrained tightly on the guidewire. Per the graphical results, the conclusions and results illustrate reduced drag and more consistent forces over multiple uses.

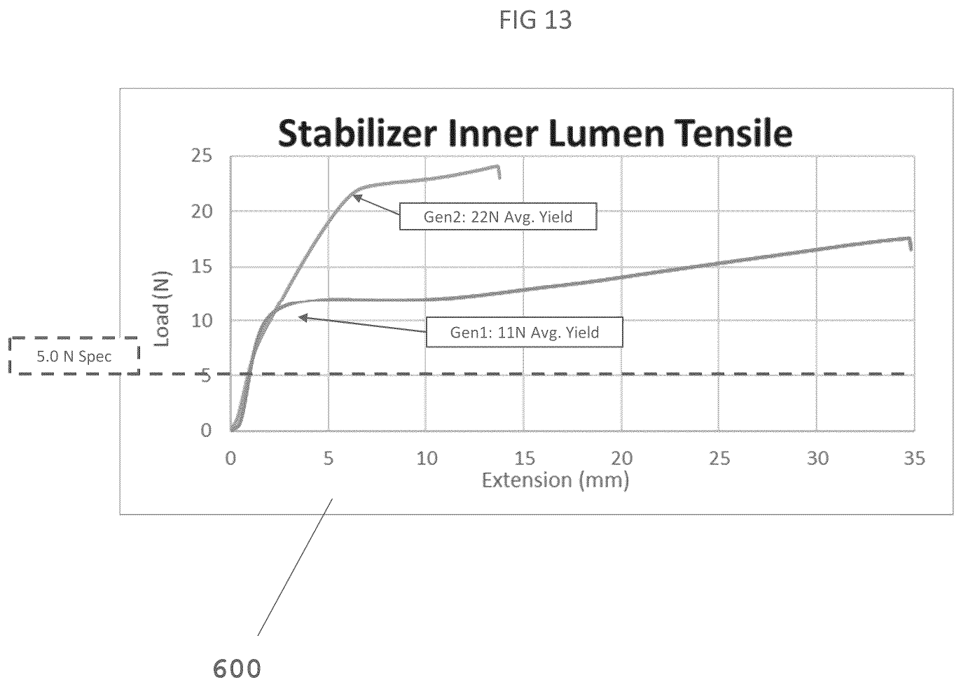

FIG. 13 illustrates a set of comparative inner lumen strength graph 600 illustrating that the GEN 2 inner lumen exhibits a Yield strength that is two times (2×) stronger than that of the GEN 1 inner lumen and an Elastic stiffness/elongation is similar. However, the GEN 2 system exhibits a thinner wall, which provides 0.0005% extra clearance for a guidewire.

Further investigating the operational enhancements from GEN 1 to GEN 2, the stabilizer improvements include improved Guidewire compatibility, increased guidewire lumen clearance (0.0005″ larger, +5%), smoother and more lubricious interior, improved abrasion resistance, 4% stronger catheter interior bonds and 2× times higher yield strength.

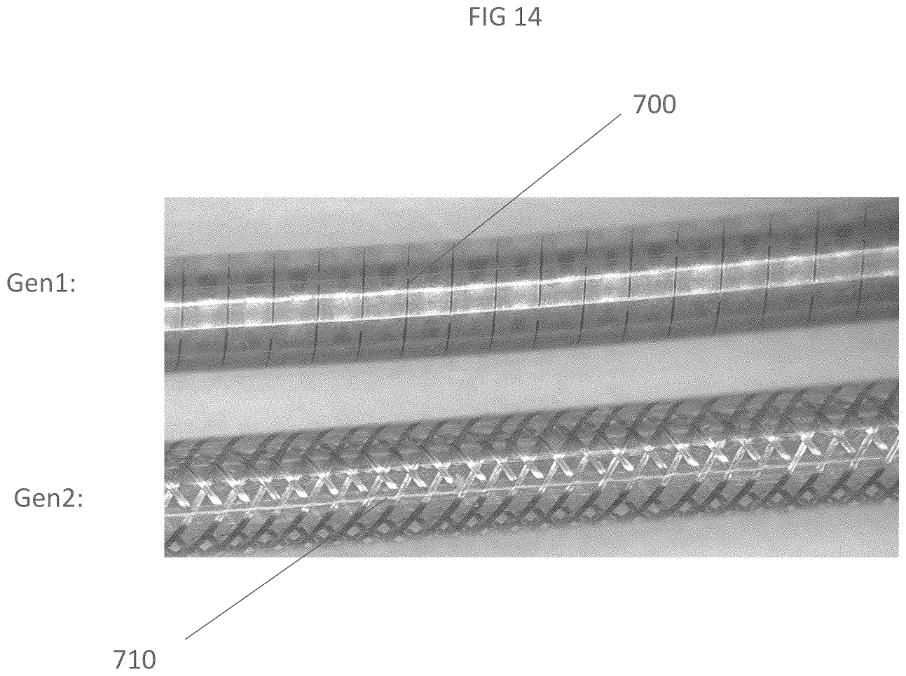

FIG. 14 illustrates the braided delivery sheath internal reinforcement advancements from GEN 1 to GEN 2. As illustrated, GEN 1 700 features, a Drawn 304SS hypotube, centerless ground OD, with a Spiral interrupt laser cut pattern. Conversely as illustrated, GEN 2 710 features, a 0.0005″×0.0025″ 304SS Flatwire braid (90 PIC) wherein a 2 over 1 pattern is employed. The system further utilizes 4× Triaxial Technora Fiber, a Vestamid Internal Layer (extruded) and a Vestamid External Layer, which is dispensed for formation,

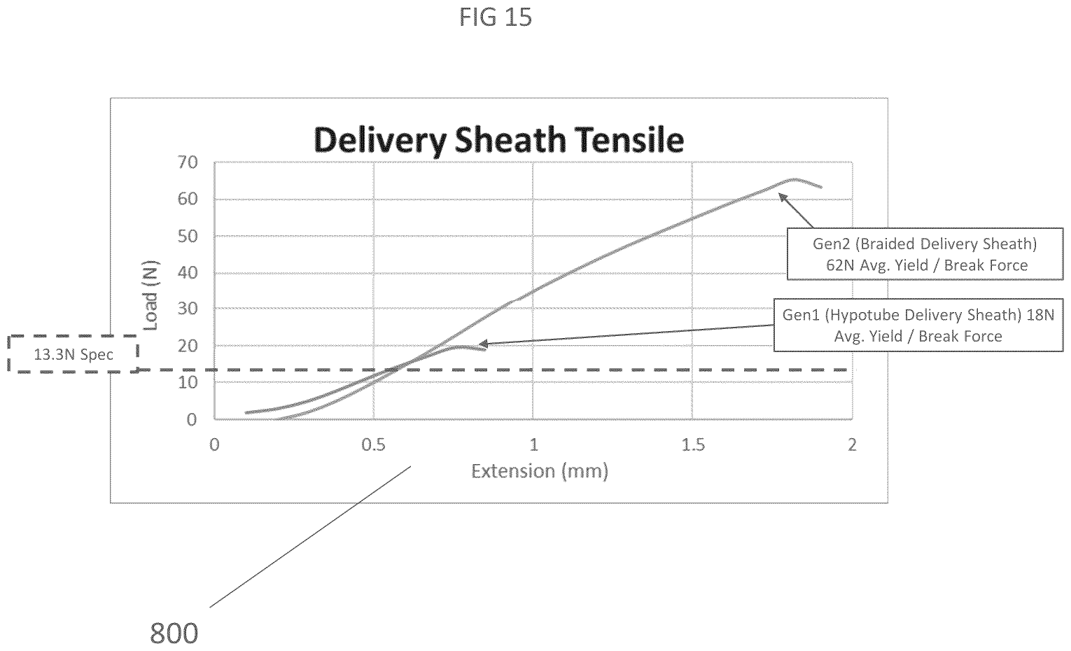

FIG. 15 illustrates graphical representation of the delivery sheath shaft tensile 800. As illustrated, when compared with Gen 1, Gen 2 features an average yield/break strength is 3.5 times stronger than that of Gen provides elastic stiffness/elongation that similar, yet provides an enhanced failure effect from kink/fracture to kink and remain attached.

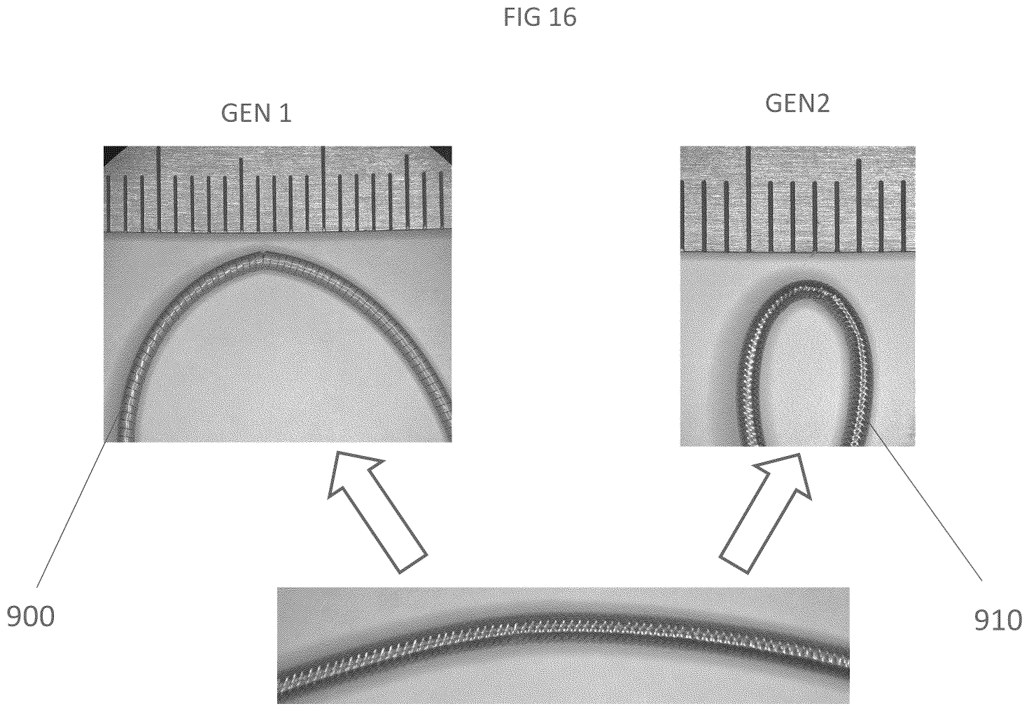

FIG. 16 illustrates the delivery sheath robustness of GEN 2 910 when compared to that of GEN 1 900 as numerous qualities are increased and several enhancements to reinforcement are exhibited in utilizing a braided skeleton tube instead laser cut hypotube. These enhanced characteristics include greater strength, and particularly 3.5× times higher break strength, higher kink resistance. The system additionally exhibits greater Robustness and higher toughness qualities through the employment of bullet proof materials which, as ancillary benefit allow for come accidental damage and axe still able to deploy the stent effectively.

In operation, the instant system is less of a finesse catheter and thus allows for deployment by a broader, and even less skilled user base. Additionally, more protection is provided for a wider range of anatomical scenarios as the exterior material has been changed to a less tacky polymey

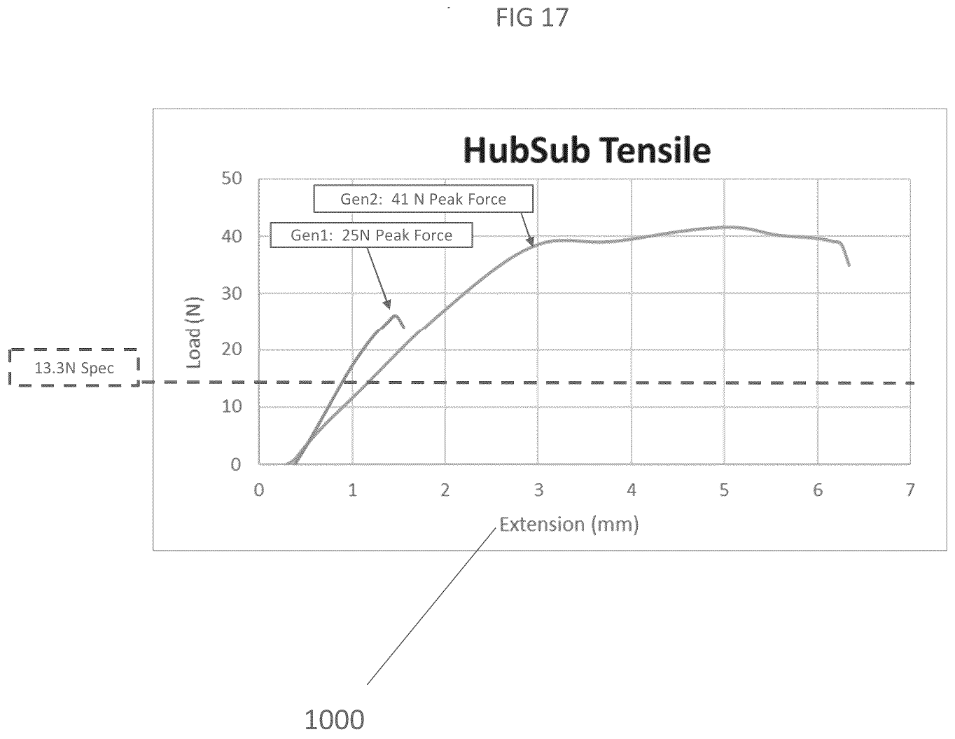

FIG. 17 illustrates the Bub Sub Tensile strength comparative graph 1000 for GEN 2 system versus the GEN 1 system. As shown in the graph of Load versus Extension, the Peak Strength of the GEN 2 configuration is 1.7 times stronger than that of the GEN 1 configuration. As indicated, the GEN 2 withstood a 41 Newton Peak Force, versus GEN 1 which withstood a 25 Newton Peak Force.

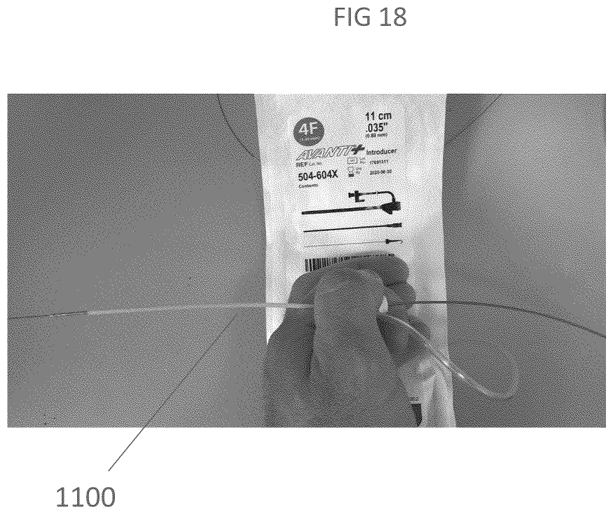

FIG. 18 illustrates GEN 2 device passing a 4Fr introducer 1100 the parameters for an overall GEN 2 delivery system, wherein access is gained using the smallest Commercially available sheath/guiding catheter which is 4F. The crossing profile of the GEN 1 system is 3.28, versus the *3.5F of GEN 2. The 3.5Fr profile was achieved by using a minimum thickness braid and fibers, a minimum thickness lamination jacket, surface not as smooth as desired and a minimum thickness PTFE Liner.

What is claimed:

1. A stent delivery system comprising:
at least one marker band comprising a portion of radiopaque material;
at least one marker band adhesive layer;
a shaft Liner;
a braided skeleton tube;
hub assembly comprises a 3.2F construction;
an extrusion delivery sheath, wherein the delivery shaft comprises a braided construction; and
a stent.

2. The stent delivery system of claim 1 further comprising a hub.

3. The stent delivery system of claim 1 further comprising at least one backflow instrument.

4. The stent delivery system of claim 3 wherein the at least one backflow instrument comprises a Tuohy Borst.

5. The stent delivery system of claim 1 further comprising a lumen comprising a single solid layer material construction which is extruded then drawn to final shape.

6. The stent delivery system of claim 1 wherein the delivery sheath is formed on a mandrel.

7. The stent delivery system of claim 1 wherein the delivery sheath comprises a 3.2F construction.

8. The stent delivery system of claim 1 wherein the at least one marker band comprises Platinum/Iridium alloy in a ration of Pt 90/Ir 10 alloy.

9. The stent delivery system of claim 1 wherein the at least one marker band comprises adhesive comprises Cyanoacrylate Loctite 4014.

10. The stent delivery system of claim 1 wherein the shaft liner comprises PTFE.

11. The stent delivery system of claim 1 wherein the shaft liner comprises etched PTFE.

12. The stent delivery system of claim 1 wherein the braided skeleton tube comprises 304 SS flatwire, and an aramid fiber.

13. The stent delivery system of claim 1 wherein the aramid fiber is selected from the group consisting of Technora and Vestamid.

14. The stent delivery system of claim 1 wherein the delivery sheath is comprised of extruded nylon Vestamid care ML21, natural.

15. The stent delivery system of claim 1 wherein the marker band comprises a Platinum/Iridium alloy.

16. The stent delivery system of claim 1 wherein the marker band comprises a Platinum/Iridium alloy in a ration of Pt 90/Ir 10 alloy.

17. The stent delivery system of claim 1 wherein the adhesive comprises CYANOACRYLATE, Loctite 4014.

18. The stent delivery system of claim 1 wherein the at least one marker band comprises a 3.2F construction.

19. The stent delivery system of claim 1 further comprising a proximally located rotational hemostasis valve on the sheath catheter provides hemostasis and a safety lock to prevent premature deployment of the stent as well as a system to irrigate the catheter.

20. The stent delivery system of claim 1 comprising a liner located internally within the stent delivery system wherein the liner exhibits tensile strength value in a range between two (2) to three (3) times the tensile strength value of industry standard hypotube liners.
